(19) United States
(12) Patent Application Publication
(10) Pub. No.: US 2021/0301319 A1
(43) Pub. Date: Sep. 30, 2021

(54) PROTEASE ASSAYS AND THEIR APPLICATIONS

(71) Applicant: CELLEX, INC., Morrisville, NC (US)

(72) Inventor: Xingxiang LI, Morrisville, NC (US)

(73) Assignee: CELLEX, INC., Morrisville, NC (US)

(21) Appl. No.: 17/213,790

(22) Filed: Mar. 26, 2021

Related U.S. Application Data
(60) Provisional application No. 63/000,045, filed on Mar. 26, 2020.
(60) Provisional application No. 63/070,027, filed on Aug. 25, 2020.

(51) Int. Cl. C12Q 1/66; C12Q 1/37; C12N 9/02; C12N 9/50

(52) U.S. Cl. CPC C12Q 1/66; C12Q 1/37; C07K 2319/50; C12N 9/503; C07K 2319/61; C12N 9/0069

(57) ABSTRACT

The application describes methods for detecting site specific proteases indicative of infection by a protease-generating pathogen. The application also describes fusion proteins for use in the methods, DNAs encoding the proteins and cells that express them. Particular applications are described including fusion proteins and methods for detecting corona viruses,.

such as SARS CoV2. Method for protease and pathogen detection described in the application include protease amplification methods and methods using inhibitors to increase sensitivity and specificity.

### CROSS-REFERENCE TO RELATED APPLICATION

This application claims priority of U.S. Provisional Application No. 63/000,045 filed on Mar. 26, 2020 and U.S. Provisional Application No. 63/070,027 filed on Aug. 25, 2020, both of which are herein incorporated by reference in their entireties.

### TECHNICAL FIELD

The present inventions relate to methods and devices for detection of pathogen protease activities and their applications including diagnostic applications for detection of certain pathogen infections in a patient or animal species. Present invention discloses a method of signal amplification enabled by a protease to be detected and its applications. The present invention further discloses methods for monitoring disease progression after infection of SARS CoV-2 virus and for diagnosis of SARS CoV-2 infection.

### GOVERNMENT FUNDING

No government funds were used in making the inventions herein disclosed.

### INTRODUCTION

Pathogens have adapted to live and replicate in their host cells and to transmit from one individual to another. Replication of a pathogen, particularly viruses, in infected human or animal hosts often rely on the nucleic acid and/or protein synthesis machines of the host cells. However, proteins encoded by the genome of a pathogen, particularly a virus, often play essential roles in the survival, replication and transmission of the pathogen. Among these pathogen-encoded proteins are endopeptidases, which are also known as proteases and proteinases.

In order to efficiently use its compact genome, viruses often synthesize the viral proteins as polyproteins, each of which contains more than one functional protein. The polyproteins must be cleaved at precise locations to release the individual proteins. In some cases, the polyproteins are cleaved by a protease of the host cells. In other cases, the viral polyproteins are cleaved by a protease encoded by viral genome itself. This latter class of viruses includes, but is not limited to, human immunodeficiency virus (HIV), human hepatitis C virus (HCV), coronavirus viruses, West Nile virus, Zika virus and dengue viruses.

Because a virus encoded protease cleaves the viral polyprotein at specific cleavage sites, viral proteases have been favorite targets for the development of antiviral drugs. Indeed, many efforts have been devoted to developing inhibitors for these viral proteases as antiviral drugs. Some of these inhibitors have become antiviral drugs. For example, protease inhibitors targeting HIV and HCV proteases are important and effective antiviral drugs. The fact that pharmaceutical drugs inhibiting viral proteases could be developed indicated that these targets are highly specific and therefore excellent target for diagnosis of a viral infection.

Fluorescent assays have been developed and used for screening viral protease inhibitors. For example, a fluorescent assay was used for screening for SARS-CoV-2 3CL enzyme inhibitors (ACS Pharmacol. Transl. Sci. 2020, 3, 5, 1008-1016). Typically, these assays use a synthetic peptide as a substrate, which contains the amino acid sequence of the cleavage site, a fluorescent moiety at one end and a quenching moiety at the other end. The quenching moiety reduces the fluorescence or causes a wavelength shift of the fluorescence. When the peptide substrate is cleaved by a protease, the fluorescence intensity increases and/or the peak wavelength changes. These changes can be measured using a fluorimeter. This type of assays is not very sensitive; but, they are adequate for inhibitor drug screening as a large amount of cloned and purified protease can be used in the screening assay. However, they are not adequately sensitive for diagnostic uses, which require the detection of a minute amount of viral protease in a sample.

Methods herein described are substantially more sensitive and hence suitable for use in diagnosis of a pathogen infection by detecting the protease activity indicative of the presence of the pathogen.

Also described herein are methods in which the protease activity being detected enables signal amplification to further improve the detection sensitivity, referred to herein as protease enabled signal amplification (PESA) technology. In embodiments which use PESA technology, a protease for which its activity is to be detected is fused through a linker containing the specific cleavage site to signal generating moiety in a recombinant protein, wherein both the protease and the signal generating moiety are inactivated in the fusion. A protease that cleaves the cleavage site in the fusion separates and activates both the protease and the signal generation moiety. In addition to the signal generated by the activated signal generation, the activated protease cleaves further fusion proteins, activating even more protease and signal generating moiety, whereby the signal is greatly amplified. In a PESA based assay, signal amplification depends on the presence of the protease in the sample and thus can be used for detection of the protease activity. If the protease activity is indicative of a pathogen or pathogen infection, the assay can be used for detection of pathogen or pathogen infection.

Current methods for diagnosis of acute infection of SARS CoV-2, which causes COVID-19 disease, are not ideal. These methods either detect viral antigen or viral nucleic acids, both of which suffer shortcomings. The antigen assays lack sensitivity, while the nucleic acid-based assays, such as PCR, use expensive equipment, require a specialized facility, and have a long wait time for results. In methods described herein all samples can be pre-screened with coronavirus protease assay, such as the 3CL enzyme assay described in greater detail below, as these assays can be rapid and highly sensitive and require simple equipment and facilities, and only positive samples are further tested with a more specific assays such as PCR.

One important feature of a COVID-19 virus infection is that a large portion of infected individuals may have mild or no symptoms while some will progress to severe clinical symptoms and even death. It is a challenge to identify those who would progress to having severe clinical symptoms and to monitor disease progress. Methods used to predict the clinical outcome are known as prognostic tests while those used to monitor disease progress or treatment effectiveness are known as monitoring tests. Currently there is no effective prognostic or monitoring test for COVID-19 disease.

Some advantages of COV-19 diagnostic technology are as follows:

For Nucleic Acid assays, the advantages are that they are easy to develop and highly sensitive. However, the sample processing is difficult, they have long turnaround times, their reagents are in short supply and they are susceptible to genetic mutations.

For Antibody/Antigen assays, the advantages are that they are easy to use and suitable for POC use. However, they can be more difficult to develop. Antibody assays rely on antibodies whose appearance can be 10-14 days after infection. They also have a lower specificity and sensitivity when compared to molecular assays and, like molecular assays, are susceptible to genetic mutations.

For Viral Enzyme Activity assays, the advantages are that they are highly sensitive, easy to use (one or two step(s) assay), suitable for large-scale production, and suitable for POC or large-scale batch testing in lab. They are also compatible with a flu assay and detect active infections only. They are also not susceptible to genetic mutations. However, they cannot differentiate between Flu A and Flu B or between COVID-19 and non COVID-19. This limitation can be resolved by subsequent confirmatory testing of positive samples using a molecular assay.

### BRIEF SUMMARY

Methods herein described use chemiluminescence or biochemiluminescence to detect protease activity in a sample. So long as the pathogen being detected contains a protease specific for a cleavage site amino acid sequence and such specificity is indicative of the pathogen, an assay can be designed for detection of the pathogen in a specific manner according to the present invention. Various embodiments are set forth illustratively in FIGS. 1-5.

In some embodiments, the protease assay is not specific for COVID-19. Instead, it detects Coronaviruses in general. The protease specificity is shared across the Coronaviruses. Therefore, in some embodiments, a second assay is required if the objective is to detect COVID-19 specifically; e.g., RT-PCR. These embodiments allow rapid, inexpensive, and accurate ways to screen patients to identify coronavirus positive individuals for testing by COVID-19 specific assays. This cuts down considerably on the need for RT-PCR tests. It may also serve as a test for Coronavirus infections generally, such as those that cause “common cold”.

Since virus infection of host cells requires the receptor of the host cells and a receptor exists in certain cell types or organs, this phenomenon can be used to monitor infection of certain organs for the purpose of disease progression monitoring. For example, COVID-19 virus (SARS-CoV-2 virus) infection requires cellular receptor ACE2 (Angiotensin Converting Enzyme 2), which abundantly exists in certain organs and cell types. These cell types include, but are not limited to, epithelium cells of the respiratory system, endothelium cells blood vessels, and tubular and glomerular epithelium in kidney. Detection of COVID-19 protease activity in urine may indicate the infection of kidney. Likewise, detection of COVID-19 protease activity in blood specimens (serum or plasma) may indicate infection of endothelium of the vascular system. Thus, the assay described in the present invention can be used to monitor infection of certain organs or tissues to monitor disease progression. This is important for monitoring kidney infection with COVID-19 virus as kidney infection may lead to acute kidney failure, a life-threatening secondary disease.

In an embodiment, as depicted in FIG. 1, a specific cleavage amino acid sequence is inserted into a protein,—the light signal enabling molecule [1]—that can enable production of a light signal [5]. In this embodiment, insertion of the protease cleavage site [4] divides the light enabling molecule into a first moiety [2] and second moiety [3]. The cleavage site insertion is designed so that it does not cause significant loss of activity of the light enabling activity of molecule unless the first and second moieties are cleaved via the specific activity of a protease [6]. Thus, when compared to the negative control sample which contains no protease activity, the loss or reduction of light signal from the sample with the protease activity is indicative of the presence of the protease and thus of pathogen or pathogen components in the sample, and infection of the host by the pathogen. In some embodiments, the light enabling molecule is a firefly luciferase.

In another embodiment, depicted in FIG. 2, the light signal enabling molecule [1] is attached to an inactivating entity [7] through a linker [4] containing a specific cleavage amino acid sequence of protease [6], which is to be detected. Little or no light signal is detected unless the linker is cleaved by the protease, thereby freeing signal enabling molecule from the inactivating entity, which increases the light signal [5]. Thus, in this type of embodiment a light signal increase in a sample being tested, compared to a negative control, is indicative of the presence of the protease activity, which in turn is indicative of the infection of the host by the pathogen. In some embodiments, the inactivating entity is a histidine tag bound to nickel coated solid phase such as a microparticle or nanoparticle. In still other embodiments, the inactivating entity is streptavidin, which can be bound to biotin coated microparticles or nanoparticles.

In another embodiment, depicted in FIG. 3, the light signal enabling molecule [1] is attached to a removable entity [8] through a linker [4] containing the specific cleavage amino acid sequence of protease [6] being detected. The linkage between the signal enabling molecule and removable entity itself does not cause inactivation of signal enabling molecule. The signal enabling molecule can be physically removed from the reaction along with removable entity unless the linker is cleaved by the protease being detected. The presence of light signal [5] in the reaction even after removal of removable entity indicates the presence of protease activity, which in turn is indicative of the infection of the host by the pathogen. In some embodiments, the removable entity is a streptavidin, which can be removed using biotin coated magnetic particles.

In another embodiment, depicted in FIG. 4, the assay is similar to what is depicted in FIG. 1 except that an additional reaction is introduced, which contains an inhibitor [9] that specifically inhibits the activity of the protease [6]. The use of an inhibitor specific for a protease can increase the specificity of the assay and allow it to distinguish different viruses of the same family, such as different Coronaviruses.

These and other embodiments of inventions herein described can be carried out in a wide variety of ways using methods well known to the art.

A variety of light enabling molecule can be used in methods herein described, including but are not limited to, various types of luciferase, phosphatase, peroxidase, and other catalysts or enzymes that directly or indirectly enable generation of a light signal. As described in embodiments herein, the light signal enabling entities can be modified to contain the specific protease cleavage amino acid sequence and cleavage of the specific site by the protease being detected causes an increase or decrease of the light signal, thereby indicating whether the donor of the sample is infected with the pathogen.

The protease indicative of a pathogen infection should cleave a specific amino acid sequence and is only present in the pathogen and/or host cells infected with the pathogen. The amino acid sequence of the cleavage site should be specific for the protease to be detected. However, the amino acid sequence may not necessarily be only one amino acid sequence; rather, it can be multiple amino acid sequences, which share similar characteristics among these sequences. The characteristics may be the hydrophobicity or size of the amino acid at certain position. The specific amino acid sequence may be any sequence which can be efficiently cleaved primarily or only by the protease to be detected. The host cellular proteases or other pathogen proteases are either deficient in the sample for detection or cannot efficiently cleave the amino acid sequence.

The sample to be tested should contain the pathogen and/or infected cells or components of the pathogen and/or infected cells.

To still increase the sensitivity of a protease dependent assay, the signal inactivating entity in a fusion protein between the signal enabling molecule and inactivating entity is a protease. When present as a fusion protein, the protease is inactivated as well. Presence of the protease activity in the sample to be detected cleaves the fusion protein at the linker site, leading to activation of both the signal enabling molecule and protease in the fusion protein. The activated signal enabling molecule produces detectable signal, directly or indirectly, indicating the presence of protease activity in the sample. In addition, the released protease from the fusion is also activated and cleaves more fusion protein, which in turn activates more signal enabling molecule and protease, leading to signal amplification. The signal amplification process is illustrated in FIG. 5.

### BRIEF DESCRIPTION OF THE DRAWINGS

FIG. 1 is a diagram depicting an embodiment in which a decrease in signal indicates the presence of a protease. A light signal enabling molecule [1] comprises moiety 1 [2] and moiety 2 [3] that together with one another to produce the light signal [ Moieties [2] and [3] are linked together by a linker [4] containing an amino acid sequence containing the cleavage site (amino acid sequence) of a protease [6], which does not cause significant loss of the light signally activity of the molecule. In the absence of the protease the light signal enabling molecule is active in producing a light signal [5]. In the presence of a protease that acts on the cleavage site, the linker is cut, moieties 2 and 3 are separated and the light signal enabling molecule is inactivated. The resulting reduction of the light signal is indicative of the presence of the protease activity that acts on the cleavage site. This, in turn, is indicative of the presence of infection of a pathogen that engenders the productions of the protease.

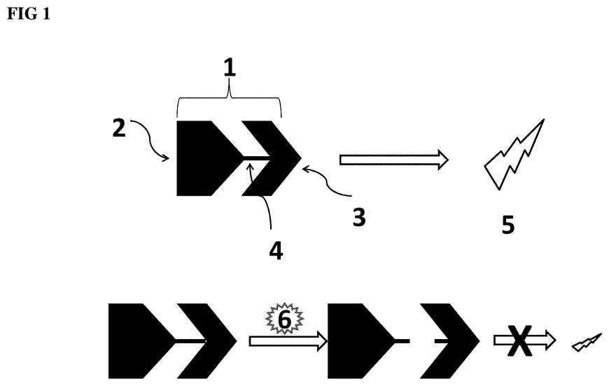

FIG. 2 is a diagram depicting an embodiment in which an increase in light is indicative of the presence of a protease. Light signal enabling molecule [1] is inactive when it is linked to an inactivating entity [7] through linker [4], which contains the cleavage site (amino acid sequence) for a protease [6]. (Other numbers are the same as for FIG. 1.) In the absence of a protease the acts on the cleavage site, the light signal enabling molecule is inactive and does not produce a light signal. In the presence of a protease that acts on the cleavage site, the linker is cleaved by the protease, resulting in dissociation of the inactivating moiety, and activation of the light signal enabling molecule. The resulting light signal and/or increased light signal is indicative of the presence of a protease that can cleave the linker, and this in turn is indicative of the presence of infection with the pathogen that engenders production of the protease.

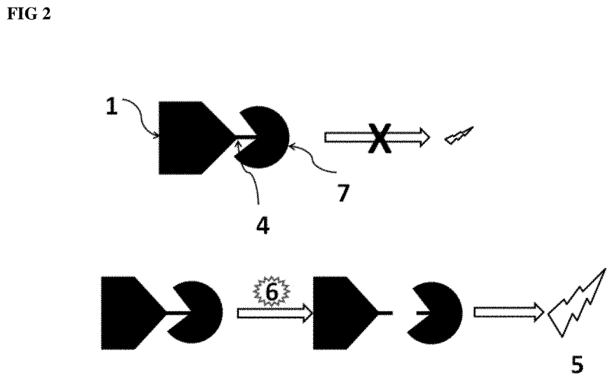

FIG. 3 is a diagram depicting an embodiment in which an increase in light production indicates the presence of a protease and, thereby, the presence of a pathogen. A light signal enabling molecule [1] is attached to a removable entity [8] through a linker [4] that contains a cleavage site (amino acid sequence) for protease [6] and by itself does not cause loss of activity of the light signal enabling molecule. (Other numbers are as in preceding Figures.) When the light signal enabling molecule is attached to removable entity is can be removed from the reaction. When the linker is cleaved by the protease being detected,. the removable entity is dissociated from the light signal enabling molecule. The dissociated light signal enabling molecule will remain in the reaction when the removable entity is removed. The resulting retention of the light signal, compared to a negative control, indicates the presence of the protease and therefore the pathogen infection. One type of removable entity is a magnetic particle, which can be removed through the use of a magnet.

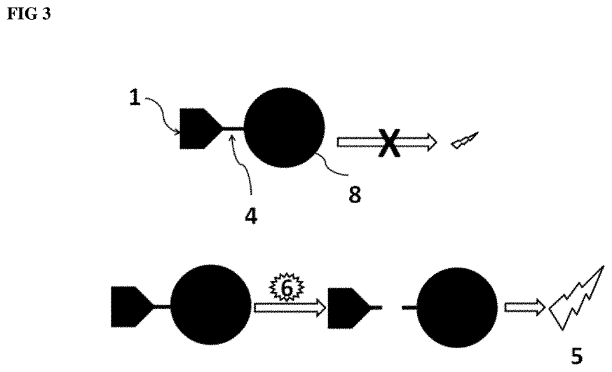

FIG. 4 is a diagram depicting the detection principle of one embodiment. In this embodiment, a protease inhibitor [9] specific for the protease being detected is used in the assay. (Other numbers are the same as in the preceding Figures.) The assay is conducted in two reactions, which are identical except that one contains the inhibitor or inhibitors. If the presence of the inhibitor or inhibitors leads to a detectable change in light signal, then the sample contains the protease being detected. Signal change can be increase or decrease of the signal. The diagram depicts a design where inhibition of the protease by the inhibitor leads to increase of the signal.

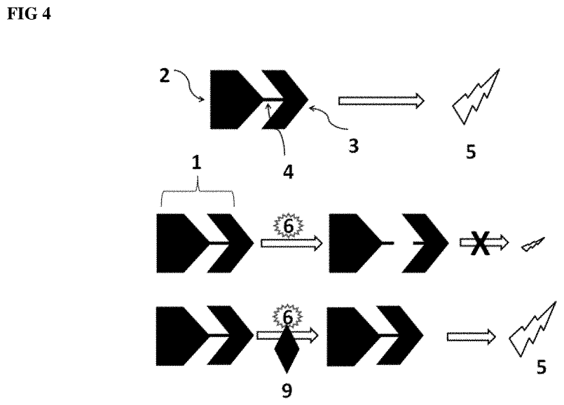

FIG. 5 is a diagram depicting an embodiment utilizing protease enabled signal amplification. A signal enabling molecule [1] is linked through a linker [4] comprising a protease cleavage site (amino acid sequence) to a protease [6]. When linked together both the signal enabling molecule and the protease are inactivated, producing little or no detectable signal. However, in the presence of protease that acts on the cleavage site, the linker is cut, releasing and activating both the signal enabling molecule and the protease. The protease thus released itself then cleaves the linker producing more signal and activating still more protease, leading to amplification of the signal. Because the protease is catalytic the amplification will be exponential.

An example of this type of embodiment is a fusion protein comprising a signal enabling protein, such as luciferase connected by a protease cleavable sequence to a viral protease, wherein the luciferase and the protease are inactive in the fusion, and regain their activity when the linker is cut.

FIG. 6 is a photograph of SDS-PAGE gel of recombinant mLuc protein digested with a recombinant 3CL enzyme. mLuc is a recombinant firefly luciferase containing a 3CL cleavage sequence.

FIG. 7 is a histogram showing the relative activity (RA) of positive controls (P1 and P2), negative control (N), and purified COVID-19 virus in serial dilutions. None of the purified virus dilution showed any 3CL activity.

FIG. 8 is a diagram depicting a fusion protein used in an embodiment of a PESA based assay. The fusion protein comprises a signal generating polypeptide region, [1], a first linker polypeptide region [4], a protease, and an optional second linker polypeptide region [10]. The first and the second linker (if present) comprise a protease cleavage site. The signal generating polypeptide is inactive when it is connected to the first linker polypeptide region in the fusion protein, and is active when it is released from the fusion protein, as by cleavage at the protease cleavage site in the linker. The protease likewise is inactive when is connected to the first linker polypeptide region in the fusion protein, active when is released from the fusion protein, as by cleavage at the protease cleavage site in the linker. The active protease released from the fusion protein recognizes and cleaves the proteases cleavage sites in the linkers in the fusion protein releasing additional active signal generating polypeptide and active protease, in a self amplifying reaction. The second linker polypeptide region decreases as much as 100% any residual activity of the protease in the fusion protein, should there be any. Additional linker with additional cleavage sites is present in additional embodiments in this regard Likewise, additional protease and signal generating entities can be incorporated into fusion proteins useful in this aspect of embodiments of inventions herein described.

### DESCRIPTION

Herein described are protease assays for research and for clinical diagnosis of an infection of a pathogen. In some embodiments the assays depend on two factors to enable specific and sensitive detection of a pathogen or an infection caused by a pathogen: a pathogen encoded protease capable of only cleaving an amino acid sequence with specific characteristics, and a light signal enabling molecule.

Many pathogens, particularly viruses, produce specific proteases, which can cleave only an amino acid sequence with specific characteristics such as amino acid sequences. The genome of human immunodeficiency virus (HIV), for example, encodes a retropep sin, which cleaves specific sequence in the HIV polyprotein. The genome of HCV encodes a NS3/NS4 serine protease that cleaves four specific sites in the HCV polyproteins.

Specific inhibitors had been identified for these proteases and used as effective antiviral medicines.

Many other viruses also have specific proteases encoded by the viral genomes. Additional examples include, but are not limited to: coronavirus, whose genomes encode encode a papain-like (PL) protease and a 3-chymotrypsin-like (3CL) protease; the dengue virus, whose genome encodes the NS2/NS3 protease; the West Nile Virus, whose genome encodes the NS2/NB3 protease; and the Zika virus, whose genome encodes the NS2B/NS3 protease.

Intense efforts are being directed to developing inhibitors targeting these proteases with the intention that will be useful as antiviral medicines.

In spite of the development of highly successful antivirals targeting these proteases, these pathogen proteases have not been used for diagnosis purposes. There are several advantages of using specific proteases for diagnosis of a pathogen infection. Pathogen proteases appear early in the infection. This means pathogen proteins, including proteases, can be detected before pathogen specific antibodies can be detected. When properly designed, detection of an enzyme activity can be more sensitive than detection of a non-enzyme protein, e.g., an antigen using a pair of antibodies. In addition, because these proteases are unique and specific for their target cleavage amino acid sequences, a homogeneous assay can be designed so that there would be no need for washing, which simplifies the assay and required instrument. Moreover, since the protease activity is essential for the life cycle of a pathogen, it is less susceptible to genetic changes, a frequent problem associated with detection of pathogens, particularly viruses. The fact that pathogen proteases are not commonly used in diagnosis of pathogen infection is primarily due to lack of sensitivity of commonly used protease assays.

Assays disclosed herein overcome these limitations. Assays are described that use light enabling molecule to generate chemiluminescence or biochemiluminescence, resulting in high sensitivity. Light enabling molecules in accordance therefore generally should be a protein or otherwise contain an amino acid sequence, that can be modified by insertion of a protease cleavage site.

In some embodiments, the light enabling molecule is a molecule enabling biochemiluminescence. One requirement of the light enabling molecule is that it can be modified to contain an amino acid sequence with the protease cleavage site without resulting in significant loss of activity. In some embodiments, cleavage of the modified molecule by the protease leads to loss of light enabling activity. In other embodiments, cleavage of the modified molecule does not lead to loss of activity, but rather, leads to loss of the capability of being physically removed from the reaction.

An example of a light signal enabling molecule is a luciferase. Many species of insects and bacteria produce luciferase to generate light, which is believed to be a mating signal in the dark. One example of insect luciferases is the firefly luciferase. Firefly luciferase can be split into two complementary moieties that can still generate light when they are in close proximity, even though they are separate entities and they lose their light-generating activity when they are separated from one another. The two moieties can be held together by a linking amino acid sequence containing a protease cleavage site without causing loss of luciferase activity. Cleavage at the cleavage site by a protease allows the two moieties to separate from one another resulting in loss of the luciferase activity.

Firefly luciferase can also be modified at its N- or C-terminus without causing loss of activity. For example, streptavidin has been fused to the N- or C-terminus of firefly luciferase. These fusion proteins normally contain extra more flexible amino acid sequence between the fused protein and luciferase. Thus, an amino acid sequence containing the protease cleavage site can be inserted between the fusion protein and luciferase. In some embodiments described herein a streptavidin-protease cleavage site-luciferase fusion protein is used for detection of the protease activity in the sample. In this assay format, the luciferase activity can be removed from the reaction using biotinylated magnetic particles unless the protease cleavage site is cleaved by the protease activity in the sample. At least one negative control is assayed along with the sample.

In an embodiment, the sample or negative control is incubated with the streptavidin-protease cleavage site-luciferase fusion protein for a period of time, followed by incubation with biotinylated magnetic particles. After removal of the magnetic particles, the remaining solution is assayed to detect luciferase activity (by adding a solution containing appropriate concentrations of ATP, DTT, CoA, Magnesium salt and luciferin). Increase in light signal in the sample as compared to that in the negative control indicates the presence of protease activity in the sample, which in turn indicates an infection of the host from whom the sample is collected.

Many potent and specific inhibitors of pathogen proteases have been developed as therapeutic drugs or drug candidates. Because a drug or drug candidate is normally highly specific for a protease of a pathogen, it can be used for specific detection of a protease or to improve the specificity of a protease assay. In an assay where a specific protease inhibitor is used, the assay is carried out in two reactions, one of which contains one or more protease inhibitor. If the protease activity is inhibited as indicated by the light signal change, then the protease is present in the sample.

In some embodiments, detection of protease activity in a sample uses a signal amplification method, which depends on the protease activity being detected, to achieve even higher sensitivity. For convenience, this signal amplification method is termed “protease enabled signal amplification”, or PESA. This technology is called PESA technology. An assay based on the PESA technology is called PESA based assay.

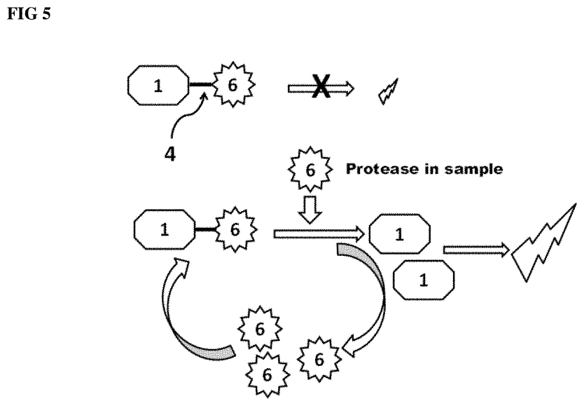

The PESA technology is best understood by referring to FIG. 5. In one embodiment, a signal enabling molecule [1] is linked in a fusion protein with a protease [7]. The linkage between the signal enabling molecule and the protease contains a specific cleavage site for a protease. When physically linked together in the fusion protein both the signal enabling molecule and protease are inactive. When a protease cleaves the fusion protein at the specific cleavage site in the linkage between the signal enabling molecule and protease both are freed from the fusion protein and thus activated. The activated protease thus liberated cleaves the recombinant fusion protein, freeing and activating additional protease and signal enabling molecule in a self-perpetuating cycle, thus amplifying the signal. All the reactions are enzymatic resulting in substantial, in some cases exponential, signal amplification.

The signal enabling molecule in the fusion for a PESA-based assay can be those enabling chemiluminescence or biochemiluminescence as described above. It can also be an entity that produces a product that can be detected by other means. Examples of these signal enabling molecules include, but are not limited to, RNA polymerases such as T7 RNA polymerase, enzymes that can convert ADP or AMP to ATP, sequence specific nucleases, kinases, nonspecific nucleases such as exonucleases, and yet another proteases.

A variety of PESA based assays can be designed.

In some embodiment, the fusion protein is a fusion between the signal enabling molecule at the N terminus and the protease at the C terminus, linked by a linker that contains a protease cleavage site. To lower the possibility of autocleavage by the protease in the fusion protein, another protease cleavage site can be introduced to the C terminus of the protease in the fusion. In still other embodiment, both the N terminus and C terminus of the fusion contain additional protease cleavage sites so that both N and C termini are flanked by additional sequences to further minimize background activity from autocleavage. An example of PESA based assay is provided in Example 4.

Embodiments of inventions herein described include a variety of fusion proteins.

One embodiment in this regard is a fusion protein comprising: (1) a first region of a signal producing polypeptide; (2) a second region of the signal producing polypeptide and (3) a linker polypeptide that connects (1) and (2) and comprises a cleavage site for a site-specific protease,

wherein the signal producing polypeptide is active in the intact fusion protein and is inactivated when the cleavage site is cut by a protease.

Another embodiment in this regard is fusion protein comprising: (1) a signal producing polypeptide; (2) a blocking polypeptide that inactivates the signal producing polypeptide in the fusion protein and (3) a linker polypeptide that connects (1) and (2) and comprises a cleavage site for a site specific protease,

wherein the signal producing polypeptide is activated when the linker is cut by a protease.

Another embodiment in this regard is a fusion protein comprising: (1) a signal producing polypeptide; (2) a site specific protease polypeptide and (3) a linker polypeptide that connects (1) and (2) and comprises a cleavage site for a site specific protease, whereby the signal producing polypeptide and the protease polypeptide both are inactive when they are connected by the linker in the fusion, and both are activated when the linker is cut by a protease.

This embodiment is a signal amplification construct. When the initial linker cleavage by a protease releases not only the signal producing polypeptide but also releases the protease which in turn cleaves the linkers in other copies of the fusion protein releasing yet more signal producing polypeptide and protease, resulting in a multiplicative amplification of the signal.

In various related embodiments, the signal producing polypeptide and/or the protease can be flanked by additional linkers in the fusion protein, to facilitate their release and activation.

Additional embodiments in these regards provide polynucleotides encoding the fusion proteins described above, including unmodified and modified RNA and DNA. Such embodiments include cloning vectors, including plasmid, bacteriophage and viral vectors of all kinds, which are known to the art.

Embodiments include cells comprising the aforementioned polynucleotides encoding fusion proteins, particularly cells for producing the fusion proteins.

### ILLUSTRATIVE EXAMPLES

The Examples below are illustrative of various aspects and embodiments of inventions herein disclosed but are in no ways limitative thereof. A complete understanding the inventions in this application is to have only by reading the entirety of the disclosure, including the claims, of the application and those of priority documents, in the context of the prior art as a whole, with the understanding of a person of skill in the art.

### Example 1: Firefly Luciferase as the Light Signal Enabling Molecue for Detection of Protease Activity of a Coronavirus

In this example, firefly luciferase is used as the light signal enabling molecule for detection of the protease activity of a coronavirus. In this biochemiluminescence reaction, D-Luciferin is oxidized by luciferase to produce light. This is one of the most efficient light production systems. It can detect as few as 2000 luciferase molecules. It is also less susceptible to interference.

Coronavirus has two proteases targeting unique amino acid sequences with 3CL as the predominant one. Firefly luciferase can be modified by inserting a 3CL cleavage site within the luciferase sequence. The cleavage of the modified luciferase leads to inactivation of the enzyme. Thus, reduction in signal compared to control indicates presence of 3CL enzyme. Additionally, 3CL is present only in infected cells, not the virus. Thus, the assay detects active infection.

The firefly luciferase gene sequence containing the 3CL cleavage site can be expressed in E. coli as a recombinant protein. This recombinant firefly luciferase containing the 3CL cleavage site is named mutant luciferase or mLuc in this example. Construction, cloning and expression of recombinant proteins in E. coli is well known to those skilled in the art. mLuc was constructed, cloned into an appropriate vector and expressed in E. coli according to methods available in the literature, using commercially available reagents.

To test whether the recombinant mutant luciferase (mLuc) with 3CL cleavage site could be properly cleaved with 3CL protease, the recombinant mLuc was mixed with a recombinant COVID-19 viral 3CL enzyme and incubated at 37° C. Aliquots were removed at 0, 5, 10, 15, 20, 25 and 30 minutes after initiating the reaction, and were stopped immediately after removal by heat inactivation in a solution with SDS. The resulting reaction solution were resolved on an SDS-PAGE gel, followed by staining to visualize the proteins on the gel.

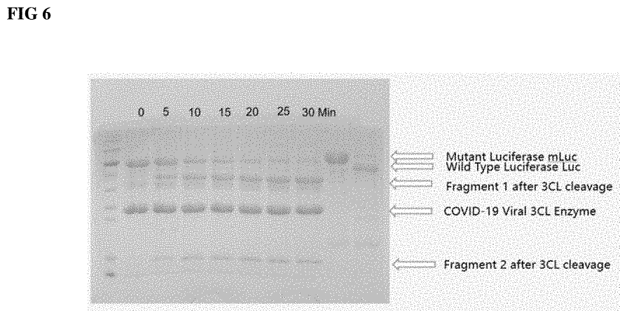

As shown in FIG. 6, significant cleavage was evident after 5 minutes. The mLuc is larger than the wild type Luc in molecular weight. Cleavage by 3CL enzyme resulted in two fragments, Fragments 1 and 2, as expected, demonstrating that mLuc can be cleaved by the 3CL enzyme.

### Example 2: a Biochemiluminescent Assay for Detection of Cronovirus 3Cl Activity

In this example, mLuc described in Example 1 was used in an assay for detection of coronavirus 3CL activity. This assay consisted of two reagents, Reagent I and Reagent II. Reagent I contained ingredients that enable 3CL cleavage while Reagent II contained ingredients that enable firefly luciferase biochemiluminescent reaction.

A commercially available feline vaccine produced in culture cells was serially diluted and used in the experiments in this example. Since the vaccine contained ingredients from the cells, the samples also contained 3CL enzyme in the sample. The samples were first mixed with Reagent I and incubated at room temperature for 15 minutes along with a negative control, which contained no 3CL enzyme. After incubation, the reactions were mixed with Reagent II and immediately placed in a luminometer to measure the light signal. Relative light units were recorded.

For comparison, the diluted samples were also tested with real time RT-PCR.

The test results are shown in Table 1. As expected, the light signal increased as the samples were more diluted, which decreased the concentration of 3CL enzyme in the samples. On the other hand, the light signal of the samples relative to the negative control (the relative activity (RA)), was inversely related to the dilution factors. Based on extrapolation, a dilution of 1:106,739 would have given an RA of 1, which is the cut off value for detecting the coronavirus assay in this example. The extrapolated Ct value at 1:106,739 dilution is equivalent to detection sensitivity of 36 cycles of real time RT-PCR.

TABLE 1
Test Results of Serially Diluted Samples
Containing the 3CL Enzyme

| | | Light | |
|---|---|---|---|
| Sample | RT-PCR | Signal | Relative |
| Dilution | (average Cₜ | (Relative | Activity |
| Factor | Value; n = 3) | Light Unit) | (RA) |
| 1:10 | 18.22 | 46 | 19421 |
| 1:100 | 21.88 | 136 | 6569 |
| 1:1000 | 25.54 | 216 | 4136 |
| 1:10000 | 27.57 | 2612 | 342 |
| Negative Control | N/A | 893,388 | 1 |

The method disclosed in this Example may be used to detect coronavirus infection. A clinical sample such as a throat swab or nasopharyngeal swab can be eluted in a sample buffer compatible with Reagent I. The ingredients in Reagent I may be prepared in, for example, 2× solution. The sample in sample buffer is mixed with 2× concentrated Reagent I in a 1:1 volume ratio. Incubate the reaction at room temperature for 15 minutes, followed by addition of equal volume of 3× Reagent II. The signal can be measured with a luminometer.

In some embodiments, the sample swabs are directly inserted into Reagent I and are left at room temperature for at least 15 minutes. The swabs are then removed from the Reagent I solution. After addition of Reagent II, the light signal is measured with a luminometer.

If the sample from a patient is positive with the assay described in the present invention, the sample proceeds immediately to confirm that positive test with a RT-PCR test to determine whether the sample contains COVID-19 virus. However, if the sample tests negative, there is no need for a RT-PCR test as the test has indicated that the patient is negative of all coronaviruses including COVID-19.

### Example 3: A Biochemiluminescent Assay for Detection of Cronovirus 3Cl Activity in Purified COVID-19 Virus

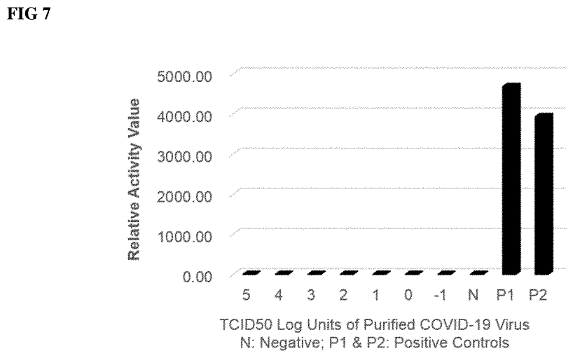

The assay described in Example 2 was used to test the samples containing serially diluted purified COVID-19 virus at concentrations ranging from 0.1 to 10⁵ TCID₅₀/mL. These virus samples were tested along with two positive controls, which contained recombinant 3CL enzyme, and one negative control. The light signal of the samples was compared to that of the negative control to derive the relative activity (RA). No 3CL activity was detected, indicating that coronavirus itself does not contain 3CL enzyme. The data is provided in FIG. 7.

### Example 4: A Protease Enabled Signal Amplification (Pesa) Assay for Detection of Cronovirus 3Cl Activity

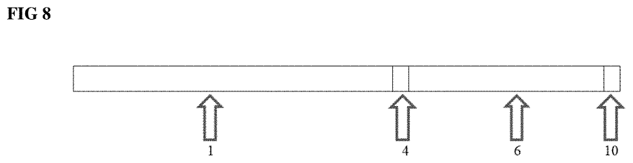

Aspects of this example are illustrated in FIG. 8.

In this example, firefly luciferase is used as the signal enabling molecule [1] and COVID-19 virus 3CL is used as the protease [6] for detection of COVID-19 virus infection. A recombinant fusion protein is constructed to contain the entire sequence of the firefly luciferase sequence in the N terminus, which is fused with the entire sequence of COVID-19 viral 3CL sequence [6] at the C terminus of the firefly luciferase [1]. Several amino acids in the virus sequence franking the N terminus of 3CL coding sequence is also introduced between the firefly luciferase and 3CL protein sequences as a linker sequence. This linker sequence [4] is derived from the COVID-19 virus pre-protein sequence. In some embodiment, this linker sequence is 4 amino acid sequence of AVLQ, which represents the amino acid sequence of alanine-valine-leucine-glutamine.

Another affinity moiety [10] can be added to the C terminus of 3CL sequence to further reduce background of 3CL enzyme in the recombinant fusion protein. Appropriate affinity moiety can be a His tag consisting of 6-8 amino acid histidine, which can bind to nickel ion. Another example of the affinity moiety is the streptavidin sequence, which can bind to biotin coated to a nanoparticle. A 3CL cleavage sequence needs to be introduced in front of affinity moiety [10] so that it can be cleaved by 3CL enzyme in the sample.

The nucleic acid sequence encoding the recombinant protein sequence can be cloned into an appropriate vector for expression in appropriate cells such as E. coli cells. After purification, the recombinant protein is used in a PESA assay for detection of 3CL activity in a sample. It is expected that there may be background activity due to low level of self-cleavage. A negative control is tested along with samples. The residual signal from the negative control is used the background signal, or commonly referred to as “noise”, to calculate the signal to noise ratio or S/N, which is the signal intensity in the sample divided by the signal intensity of the negative control. Presence of 3CL enzyme in the sample is indicated when the signal to noise or S/N exceeds 1.5, 2.0, 3.0, 4.0 or 5.0 or another threshold value.

What is claimed is:

1. A method for detection of a protease activity indicative of an infection by a pathogen in a host from which the sample is collected, the method comprising:
a. Contacting the sample with a reaction mixture containing a light signal enabling molecule modified with an amino acid sequence that can be specifically cleaved with a protease being detected and other components and conditions sufficient to enable light signal production;
b. Contacting a negative control sample with the same reaction mixture as in a,
c. Optionally contacting the sample with a second reaction mixture containing a specific inhibitor for the protease;
d. Incubating a and b for a certain period of time;
e. Measuring the signal in a and b;
f. Determining whether the protease activity is present in the sample by comparing signal change between a and b, wherein a change in signal intensity above a cutoff value indicates an infection by the pathogen;
g. Or determining whether the protease activity is present in the sample by comparing signal change between a and c, wherein a change in signal intensity above a cutoff value indicates an infection by the pathogen.

2. The method of claim 1, wherein specific cleavage of the signal enabling molecule by the protease leads to change in signal intensity as compared to that of negative control, thereby indicating an infection of the pathogen.

3. The method of claim 1, wherein specific inhibition of the protease activity leads to change in signal intensity as compared to that without an inhibitor, thereby indicating an infection of the pathogen.

4. The method of claim 1, wherein the pathogen to be detected is human immunodeficiency virus (HIV), human hepatitis virus type C (HCV), coronavirus (CoV), dengue virus (DENV), West Nile virus (WNV), Zika virus, or any other virus encoding a viral protease able to cleave a specific amino acid sequence.

5. The method of claim 4, wherein the protease is any one or more of the HIV retropepsin protease (also known as the HIV retroviral aspartyl protease), the HCV NS3/NS4 serine protease, the coronavirus chymotrypsin-like (3CL) protease or papain, the dengue virus NS2/NS3 protease, the West Nile Virus NS2/NB3 protease, and the Zika virus NS2B/NS3 protease;

6. The method of claim 1, wherein the protease specific amino acid sequence is inserted within the light signal enabling molecule, wherein the insertion does not cause significant loss of the light signal enabling activity of the molecule.

7. The method of claim 6, wherein cleavage of the protease specific amino acid sequence in the signal enabling molecule by protease in a sample causes loss or decrease in the light signal intensity compared to a negative control sample, thereby indicating a pathogen infection in the host.

8. The method of claim 1, wherein the protease specific amino acid sequence is comprised in a linker sequence fused to the N or C terminus of the light signal enabling molecule on one end and to an inactivating moiety on the other end, whereby the light signal enabling molecule is inactivated.

9. The method of claim 8, wherein cleavage of the linker by the protease leads to recovery of the activity of light enabling molecule;

10. The method of claim 9, wherein increase of light signal in a reaction indicates the presence of protease in the sample and thereby indicates a pathogen infection in the host.

11. The method of claim 1, wherein the signal enabling molecule is linked to removable entity through a linker containing a cleavage site of a protease indicative of a pathogen infection.

12. The method of claim 11, wherein the removable entity can be removed from the reaction along with the signal enabling molecule unless the linker is cleaved by the protease in a sample.

13. The method of claim 11, wherein the loss of activity of the signal enabling molecule in the reaction is indicative of the presence of protease in the sample and thereby indicate a pathogen infection of the host.

14. The method of claim 1, wherein the light signal enabling molecule is a luciferase, a peroxidase, or an alkaline phosphatase.

15. The method of claim 14, wherein the luciferase is a firefly luciferase, a click beetle luciferase or a bacterial luciferase

16. The method of claim 1, wherein the protease specific amino acid sequence comprises a sequence of at least three amino acids.

17. The method of claim 16, wherein the protease specific amino acid sequence is flanked by additional amino acids not needed for recognition by the protease.

18. The method of claim 1, wherein the pathogen is a coronavirus.

19. The method of claim 18, wherein the coronavirus proteases used for coronavirus infection detection are the coronavirus papain protease and/or 3CL protease.

20. A method for detection of a protease activity indicative of an infection by a pathogen in a host from which the sample is collected, comprising.
a. Contacting a sample with a reaction mixture containing a recombinant fusion protein of a signal enabling molecule linked to a protease through a linkage with an amino acid sequence that can be specifically cleaved by a protease being detected;
b. Contacting a negative control sample with the same reaction mixture as in a,
c. Optionally contacting the sample with a second reaction mixture containing a specific inhibitor of the protease;
d. After incubation for a certain period of time, measuring the signal in a and b;
e. Comparing signal change between a and b, wherein a change in signal intensity above a cutoff value indicates the presence of the protease and an infection by the pathogen;
f. Or determining whether the protease activity is present in the sample by comparing the signals measured for a and c, wherein a change in the signal intensity above a cutoff value indicates the presence of the protease and an infection by the pathogen.

21. The method of claim 20, wherein the intact fusion protein has very little or no signal enabling or protease activity and cleavage of the linkage by a protease in the sample activates both the signal enabling molecule and protease from the fusion protein.

22. The method of claim 21, wherein the activated protease from the fusion protein in turn cleaves more fusion protein and activating both more signal enabling molecules and more protease, thus amplifying the signal generated by the signal enabling molecule.

23. The method of claim 20, wherein the signal enabling molecule is a light signal enabling molecule, a fluorescence enabling molecule, or an enzyme that produces a detectable product detectable product.

24. The method of claim 20, wherein the protease is encoded by a pathogen gene and can cleave a specific amino acid sequence, whereby the protease activity is indicative of an infection by the pathogen.

25. A method for detection of SARS COV-2 infection, the method comprising:
a. Detection of a sample with a coronavirus protease assay first and, if positive,
b. Detection of the sample with a second test specific for SARS CoV-2 infection or a second sample is collected from the same individual and tested with a second test specific for SARS CoV-2.

26. The method of claim 25, wherein the coronavirus protease is the papain like protease or 3CL.

27. The method of claim 25, wherein the second test specific for SARS CoV-2 infection is a RT-PCR based assay using specific primers or an antigen assay using specific antibodies.

28. A fusion protein comprising: (1) a first region of a signal producing polypeptide; (2) a second region of the signal producing polypeptide and (3) a linker polypeptide that connects (1) and (2) and comprises a cleavage site for a site-specific protease, wherein the signal producing polypeptide is active in the intact fusion protein and is inactivated when the cleavage site is cut by a protease.

29. A DNA encoding the fusion protein according to claim 28.

30. A cell comprising a DNA according to claim 29.

31. A fusion protein comprising: (1) a signal producing polypeptide; (2) a blocking polypeptide that inactivates the signal producing polypeptide in the fusion protein and (3) a linker polypeptide that connects (1) and (2) and comprises a cleavage site for a site specific protease, wherein the signal producing polypeptide is activated when the linker is cut by a protease.

32. A DNA encoding the fusion protein according to claim 31

33. A cell comprising a DNA according to claim 32

34. A fusion protein comprising: (1) a signal producing polypeptide; (2) a site specific protease polypeptide and (3) a linker polypeptide that connects (1) and (2) and comprises a cleavage site for the site specific protease, whereby the signal producing polypeptide and the protease polypeptide both are inactive when they are connected by the linker in the fusion protein, and both are activated when the linker is cut by the protease.

35. A fusion protein according to claim 34, further comprising an additional linker polypeptide at the end of the protease polypeptide distal to linker polypeptide connecting the signal producing polypeptide to the protease polypeptide.

36. A DNA encoding the fusion protein according to claim 34.

37. A cell comprising a DNA according to claim 36.
